(19) United States
(12) Patent Application Publication
(10) Pub. No.: US 2011/0152653 A1
(43) Pub. Date: Jun. 23, 2011

(54) SYSTEMS AND METHODS FOR IMPLEMENTING RAPID RESPONSE MONITORING OF BLOOD CONCENTRATION OF A METABOLITE

(75) Inventors: Avraham Shekalim, Nesher (IL); Noam Peleg, Gan-Ner (IL); Erez Yotvat, Kibbutz Ein Carmel (IL)

(73) Assignee: G-Sense Ltd., Tirat Hacarmel (IL)

(21) Appl. No.: 12/996,419

(22) PCT Filed: Jun. 7, 2009
(86) PCT No.: PCT/IL09/00566

§ 371 Date: Dec. 6, 2010

Related U.S. Application Data
(60) Provisional application No. 61/058,935, filed on Jun. 5, 2008.

(51) Int. Cl. A61B 5/1468

(52) U.S. Cl. 600/345

(57) ABSTRACT

Systems and methods for monitoring the concentration of glucose or other metabolites by way of a low-volume microdialysis-probe (10) operative in accordance with a pulsed mode of flow through a flow path (12) defined by a layered structure. Also described are a system and a method for deployment of the microdialysis probe (10) within the body and a technique for deriving a metabolite concentration of the basis of a rate of change of an optical parameter.

### FIELD AND BACKGROUND OF THE INVENTION

The present invention relates systems and methods for monitoring the concentration of glucose or other metabolites in the blood and, in particular, it concerns a system employing a microdialysis probe.

The following documents are indicative of the state of the relevant art as known to the Applicant at the time of filing this application: U.S. Pat. No. 5,735,832; U.S. Pat. No. 5,741,284; U.S. Pat. No. 5,735,832; U.S. Pat. No. 6,805,683; U.S. Pat. No. 5,372,582; U.S. Pat. No. 5,106,365; U.S. Pat. No. 4,694,832; U.S. Pat. No. 3,830,106; U.S. Pat. No. 5,706,806; PCT Patent Application Publication No. WO 07/048786; U.S. Pat. No. 7,008,398; U.S. Pat. No. 5,002,054; U.S. Pat. No. 6,607,511; U.S. Pat. No. 4,755,173; U.S. Pat. No. 5,372,582; U.S. Pat. No. 3,359,978; U.S. Pat. No. 6,572,566; and PCT Patent Application Publication No. WO 2008/056363.

One critical issue in wide scale application of blood glucose monitoring using microdialysis probes is the time lag between changes in the actual blood glucose level and sensing of the changed values. The time-line progression of dialysate solution in a microdialysis-based metabolite monitoring system from introduction into the microdialysis probe until a measurement is obtained may be described in general terms as follows:
Diffusion time: the dialysate must be present in the microdialysis probe for a dwell time sufficient to absorb the metabolite from the surrounding body fluids through the semi-permeable membrane and reach equilibrium.
Transport time: the dialysate must be transported from the probe to the measurement volume where the metabolite concentration is to be measured.
Reaction time: the time taken for the metabolite in the dialysate to undergo a chemical reaction with a reagent to facilitate measurement of the metabolite concentration.

Of these components, the reaction time may be regarded as a fixed time delay which cannot be altered. The diffusion time is primarily a function of the surface-to-volume ratio of the probe and of the membrane properties. The transport time is a function of the transport volume V₀ of the system between the probe and the measurement volume, and of the flow rate of the dialysate. In existing systems, an excessive transport time has been found to be the most troublesome problem hampering implementation of a rapid response glucose monitoring system based on microdialysis.

In a continuous flow system, the maximum flow rate is defined by the required diffusion time within the probe. For a small probe volume, preferred both for ease of insertion and for large surface-to-volume ratio, the flow rate must be sufficiently small to ensure the required dwell time of dialysate within the probe. This in turn leads to a prolonged transport time as the slow flow rate transfers the metabolite-rich dialysate through the relatively large V₀, resulting in considerable time lag in measurements.

While it would be advantageous to reduce the transport volume V₀ in order to decrease this time lag, existing systems have achieved limited success in doing so. Specifically, since the measurement system and the probe typically cannot be manufactured as a single unit, the flow path from the probe to the measurement volume invariably has considerable length and includes a connector. The presence of a connector typically requires a relatively large gauge conduit on at least one side of the connection.

A further shortcoming of the presence of a connector is that part of the sample or an air bubble may become lodged at the connector, particularly where a change of internal diameter occurs, interfering with the fluid flow and producing a “dead volume”. A similar problem is created by systems which employ concentric tubes for forward and reverse flow where the annular channel between the tubes may lead to air bubbles becoming lodged at one side while the fluid flow passes them by on the other side, thereby creating a dead volume.

A further consideration in development of a practical blood glucose monitoring system is the volume of dialysate required. Particularly for body-mounted systems which are intended to be worn with minimum intrusiveness to the daily routine of the user, the storage volume for dialysate and reagent are severely restricted. It is therefore desirable to reduce the probe volume, as well as the transport volume, as much as possible in order to reduce the required dialysate flow rates. However, reduction in dimensions of the microdialysis probe carries with it complications of kinking or buckling of the probe membrane, possibly leading to flow obstruction or unreliable results.

There is therefore a need for a metabolite monitoring system based on a microdialysis probe which would provide rapid measurement of blood metabolite levels while minimizing the required fluid flow rates of dialysate and reagent.

### SUMMARY OF THE INVENTION

The present invention is a system and method based on a microdialysis probe for monitoring the concentration of glucose or other metabolites in the blood.

According to the teachings of the present invention there is provided, a method for monitoring the concentration of a metabolite in vivo within a body fluid, the method comprising the steps of: providing a monitoring device comprising: (i) a microdialysis probe formed at least in part from a membrane permeable to the metabolite, the probe defining a probe volume containing dialysate, (ii) a measuring cell having a measuring volume, and (iii) a flow path connecting from the probe volume to the measuring volume; (b) bringing the probe into contact with the body fluid; (c) maintaining substantially zero flow conditions during a diffusion period to allow diffusion of the metabolite into the dialysate within the microdialysis probe; and (d) generating a dialysate flow to carry a quantity of dialysate from the probe volume along the flow path to the measuring volume, wherein the generating a dialysate flow is performed such that the quantity of dialysate passes from the probe volume to the measuring volume in a given transport time, the transport time being no more than 25 percent of the diffusion period.

There is also provided according to the teachings of the present invention a device for monitoring the concentration of a metabolite in vivo within a body fluid, the device comprising: (a) a microdialysis probe for bringing into contact with the body fluid, the probe being formed at least in part from a membrane permeable to the metabolite, the probe defining a probe volume containing dialysate; (b) a measuring cell having a measuring volume; (c) a flow path connecting from the probe volume to the measuring volume; and (d) a fluid flow controller deployed to control flow of dialysate through the probe volume and the flow path to the measuring volume, the fluid flow controller being configured to generate a pulsed flow pattern including: (i) a diffusion period of substantially zero flow conditions to allow diffusion of the metabolite into the dialysate within the probe; and (ii) a dialysate flow to carry a quantity of dialysate from the probe volume along the flow path to the measuring volume, wherein the flow controller generates the dialysate flow such that the quantity of dialysate passes from the probe volume to the measuring volume in a given transport time, the transport time being no more than 25 percent of the diffusion period.

According to a further feature of the invention wherein the transport time is no more than 10 percent of the diffusion period.

According to a further feature of the invention, wherein the substantially zero flow conditions and the dialysate flow are generated repeatedly as part of a pulsed flow pattern, and wherein a single fluid pulse of the pulsed flow pattern transports the quantity of dialysate from the probe volume to the measuring volume.

According to a further feature of the invention, wherein at least part of the flow path from the probe volume to the measuring volume is formed as a channel enclosed between at least two layers of a layered structure.

According to a further feature of the invention, wherein the layered structure further comprises a reagent inlet in flow connection with the flow path for introducing a reagent for mixing with the dialysate from the probe prior to the dialysate reaching the measuring volume.

According to a further feature of the invention, wherein the measuring volume is enclosed between layers of the layered structure.

According to a further feature of the invention, wherein at least one of the layers enclosing the measuring volume is a transparent layer, and wherein the device further comprises an optical sensor for sensing an optical parameter of fluid within the measuring volume via the at least one transparent layer.

According to a further feature of the invention, wherein the probe comprises a length of flexible tube formed primarily from the membrane permeable to the metabolite, each end of the length of flexible tube being in fluid connection with an aperture formed in at least one layer of the layered structure.

According to a further feature of the invention, wherein the layered structure further comprises an insertion bore aligned with the probe for insertion of an inserter to support the probe during insertion into the body of a subject.

According to a further feature of the invention, there is also provided an inserter comprising: (a) an elongated shaft having a penetrating tip and a recess for receiving a part of the probe; and (b) an actuator element, deployed at least partially coextensively with the elongated shaft, wherein the elongated shaft and the actuator element are configured such that, in a first relative position of the elongated shaft and the actuator element, the inserter retains the probe for insertion through a biological barrier into tissue, and, when the actuator element is displaced relative to the elongated shaft, the probe is released, thereby allowing withdrawal of the inserter from the tissue while the probe remains inserted within the tissue.

According to a further feature of the invention, there is also provided (a) an optical sensor deployed for sensing an optical parameter of fluid within the measuring volume; and (b) a concentration calculator operationally connected to the optical sensor, the concentration calculator comprising at least one processor, the concentration calculator being configured to derive a rate of change of the optical parameter of the fluid within the measuring volume under substantially zero flow conditions, and to calculate a concentration of the metabolite within the body fluid based at least in part on the rate of change.

There is also provided according to the teachings of the present invention, a device for monitoring the concentration of a metabolite in vivo within a body fluid, the device comprising: (a) a microdialysis probe for bringing into contact with the body fluid, the probe being formed at least in part from a membrane permeable to the metabolite, the probe defining a probe volume containing dialysate; (b) a measuring cell having a measuring volume; and (c) a flow path connecting from the probe volume to the measuring volume, wherein at least part of the flow path from the probe volume to the measuring volume is formed as a channel enclosed between at least two layers of a layered structure.

According to a further feature of the present invention, wherein the layered structure further comprises a reagent inlet in flow connection with the flow path for introducing a reagent for mixing with the dialysate from the probe prior to the dialysate reaching the measuring volume.

According to a further feature of the present invention, wherein the measuring volume is enclosed between layers of the layered structure.

According to a further feature of the present invention, wherein at least one of the layers enclosing the measuring volume is a transparent layer, and wherein the device further comprises an optical sensor for sensing an optical parameter of fluid within the measuring volume via the at least one transparent layer.

According to a further feature of the present invention, wherein the probe comprises a length of flexible tube formed primarily from the membrane permeable to the metabolite, each end of the length of flexible tube being in fluid connection with an aperture formed in at least one layer of the layered structure.

According to a further feature of the present invention wherein the layered structure further comprises an insertion bore aligned with the probe for insertion of an inserter to support the probe during insertion into the body of a subject.

According to a further feature of the present invention there is also provided, an inserter comprising: (a) an elongated shaft having a penetrating tip and a recess for receiving a part of the probe; and (b) an actuator element, deployed at least partially coextensively with the elongated shaft, wherein the elongated shaft and the actuator element are configured such that, in a first relative position of the elongated shaft and the actuator element, the inserter retains the probe for insertion through a biological barrier into tissue, and, when the actuator element is displaced relative to the elongated shaft, the probe is released, thereby allowing withdrawal of the inserter from the tissue while the probe remains inserted within the tissue.

There is also provided according to the teachings of the present invention, method for deploying a microdialysis probe, the probe being part of a device for monitoring the concentration of a metabolite in vivo within a body fluid, the method comprising the steps of: (a) providing a microdialysis probe comprising a length of flexible tube formed primarily from a membrane permeable to the metabolite, the length of flexible tube having a first end in fluid connection with a supply of dialysate, a second end in fluid connection with a measuring cell, and a substantially constant internal cross-sectional area along the length; (b) engaging the probe with an inserter, the inserter comprising a penetrating tip for penetrating through a biological barrier, and a probe support for supporting at least part of the probe; (c) introducing the inserter with the probe through a biological barrier into tissue; and (d) withdrawing the inserter from the tissue while leaving the length of flexible tube at least partially deployed within the tissue.

According to a further feature of the present invention, wherein the inserter comprises: (a) an elongated shaft providing the penetrating tip and having a recess for receiving a part of the probe; and (b) an actuator element, deployed at least partially coextensively with the elongated shaft, wherein engagement of the probe with the inserter is maintained while the elongated shaft and the actuator element are in a first relative position, and wherein, prior to the withdrawing, the actuator element is displaced relative to the elongated shaft to release engagement of the probe.

There is also provided according to the teachings of the current invention, a system for deploying a microdialysis probe as part of a device for monitoring the concentration of a metabolite in vivo within a body fluid, the system comprising: (a) a microdialysis probe comprising a length of flexible tube formed primarily from a membrane permeable to the metabolite, the length of flexible tube having a first end in fluid connection with a supply of dialysate, a second end in fluid connection with a measuring cell, and a substantially constant internal cross-sectional area along the length; (b) an inserter comprising: (i) an elongated shaft having a penetrating tip and a recess for receiving a part of the probe, and (ii) an actuator element, deployed at least partially coextensively with the elongated shaft, wherein the elongated shaft and the actuator element are configured such that, in a first relative position of the elongated shaft and the actuator element, the inserter retains the probe for insertion through a biological barrier into tissue, and, when the actuator element is displaced relative to the elongated shaft, the probe is released, thereby allowing withdrawal of the inserter from the tissue while the probe remains inserted within the tissue.

### BRIEF DESCRIPTION OF THE DRAWINGS

The invention is herein described, by way of example only, with reference to the accompanying drawings, wherein:

FIG. 1 is a general block diagram depicting a metabolite monitoring system based on a microdialysis probe, constructed and operative according to the teachings of the present invention.

FIGS. 2 and 3 are isometric top and bottom views, respectively, of a layered structure for use in the monitoring system of the present invention.

FIG. 3 is an isometric, partially cut-away view of the layered structure of FIG. 2.

FIG. 5 is an isometric cross-sectional view of the layered structure of FIG. 2.

FIG. 6 is a enlarged view of the region of FIG. 5 designated VI, showing the probe connection.

FIG. 7 is an exploded view of the layered structure of FIG. 2.

FIG. 8 is an isometric view of a first embodiment of a microdialysis-probe inserter, constructed and operative according to a further aspect of the present invention.

FIGS. 9 and 10 are enlarged isometric views of the probe inserter of FIG. 8 depicting the tip in unbiased and biased states, respectively.

FIG. 11 is a schematic, top view of the probe inserter of FIG. 8.

FIGS. 12 and 13 are schematic, cross-sectional views of the inserter of FIG. 11 depicting the tip in unbiased and biased states, respectively.

FIG. 14 is a schematic, cross-sectional view of the inserter of FIGS. 8-13 loaded with microdialysis probe.

FIG. 15 is an overall isometric view of a second embodiment of a microdialysis-probe inserter loaded with the probe.

FIG. 16 is an isometric, enlarged view of the microdialysis probe of FIG. 15 engaged in the inserter.

FIGS. 17 and 18 are schematic, cross-sectional side-views depicting the microdialysis probe inserted in the inserter in non-locked and locked states, respectively.

FIGS. 19-21 are isometric views of the probe and inserter of FIGS. 15-18 depicting non-inserted, partially inserted and fully inserted states, respectively.

FIG. 22 is an isometric view of a single-element inserter loaded with a microdialysis probe.

FIG. 23 is a schematic, partial-top view of the inserter of FIG. 22 depicting a piercing tip and a probe notch.

FIGS. 24 and 25 are schematic, cross-sectional side-views of the inserter of FIG. 23 in unloaded and loaded states, respectively.

FIGS. 26 and 27 are transmittance graphs depicting transmittance as a function of time for a number of metabolite samples.

FIGS. 28 is a flow chart for an algorithm employed for converting transmittance measurements into a metabolite concentration values.

### DESCRIPTION OF THE PREFERRED EMBODIMENTS

The present invention is a system and method based on a microdialysis probe for monitoring the concentration of glucose or other metabolites in the blood.

The principles and operation of systems and methods according to the present invention may be better understood with reference to the drawings and the accompanying description.

The present invention includes a number of primary aspects, each of which is believed to be of patentable significance in its own right, and which are most preferably used in synergy to provide a particularly advantageous combination, as will become clear. A first aspect of the invention relates to a particular mode of pulsed flow operation which is effective to greatly reduce the time lag in metabolite concentration measurement. A second aspect relates to a structure of the device which provides very low-volume flow paths from the probe to the measurement volume. A third aspect of the invention relates to a particularly preferred low-volume microdialysis probe structure and techniques for deploying such a probe in body tissue. A fourth aspect of the invention relates to a technique for deriving metabolite concentration on the basis of a rate of change of an optical parameter. Each of these aspects will now be described in turn.

### System Overview

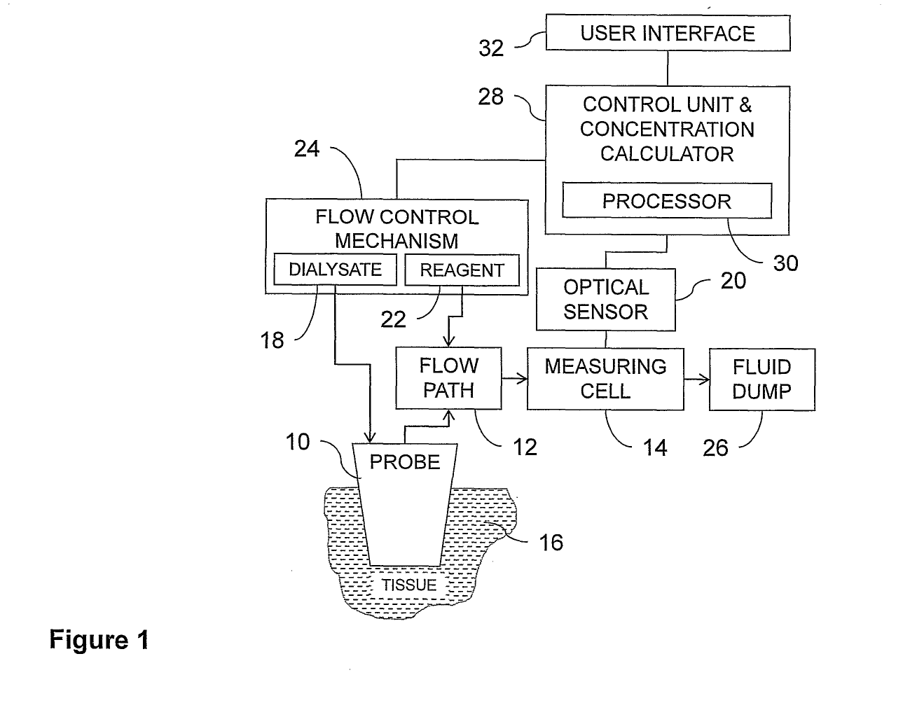

Before addressing these aspects of the present invention, it should be noted that the various aspects of the present invention will be described in the context of a microdialysis system for monitoring the concentration of a metabolite in vivo within a body fluid as illustrated schematically in FIG. 1. In general terms, embodiments of the invention include a microdialysis probe 10 formed at least in part from a membrane permeable to the metabolite in question. A flow path 12 connects from the probe volume to a measuring cell 14 having a measuring volume. In use, the probe is introduced into tissue 16 of the subject and a quantity of dialysate is supplied from a dialysate reservoir 18 to the probe, where it absorbs metabolite from the surrounding body fluids through the membrane. The dialysate is carried along flow path 12 to measuring cell 14 where the concentration of metabolite in the dialysate is determined, and the concentration in the body fluid is deduced. Any suitable technique may be used to determine the metabolite content of the dialysate reaching the measuring cell, such as various well known optical and electrical techniques. According to a particularly preferred option, measurement may be based on an optical sensor 20 sensing of a color change resulting from mixing the dialysate with a reagent (from reagent reservoir 22), although the invention is not limited to use of such a technique except where explicitly stated.

Flow of the dialysate to probe 10 and of reagent to flow path 12 for mixing with the metabolite-rich dialysate are controlled by a flow control mechanism 24. The dialysate and reagent mix in flow path 12 and then enter measuring cell 14, displacing the previously sampled fluid which drains to a fluid dump 26. Flow control mechanism 24 may be any suitable flow control mechanism, including but not limited to, various active displacement pump systems, and various arrangements of valves controlling flow from pressurized reservoirs, all as are known in the art. One particularly preferred but non-limiting example of a suitable flow control mechanism may be found in PCT Patent Application Publication No. WO 2008/056363, which is hereby incorporated in its entirety as if set out herein.

Operation of flow control mechanism 24 and processing of outputs from optical sensor 20 are typically controlled by a control unit combined with a concentration calculator 28, including a processor 30, although these functions may be separated. Control of the device and/or output of the derived metabolite concentration values are typically via a local or remote user interface 32. Additionally, or alternatively, the device may interface directly with another device, such as a drug delivery device, which takes the metabolite concentration measurements as an input.

For the sake of conciseness, the description herein will focus on features of the various aspects of the present invention believed to be relevant to the novel aspects of the invention. Additional technical details which are well known in the art and do not pertain directly to the novel aspects of the invention will not be described in detail, and may be found in prior publications, such as those mentioned in the background section of this document and the aforementioned WO 2008/056363.

### Pulsed Operation

According to one aspect of the present invention, the aforementioned problems of time lag due to the transport time can be ameliorated by use of pulsed dialysate flow where the pulse volume is coordinated with the system volume. Specifically, in one preferred implementation, substantially zero flow conditions are maintained during a diffusion period to allow diffusion of the metabolite into the dialysate within the microdialysis probe. A dialysate flow is then generated by a fluid flow controller (typically, flow control mechanism 24 operating under control of control unit 28) to carry a quantity of dialysate from the probe volume along the flow path to the measuring volume. According to a particularly preferred implementation of this aspect of the invention, the dialysate flow is generated in such a manner that the required quantity of dialysate passes from the probe volume to the measuring volume in a transport time which is no more than 25 percent of the diffusion period. Typically, the transport time is a much smaller proportion of the diffusion period, preferably less than 10% thereof, and more preferably less than 5% thereof. The total transport time is preferably no more than half a minute, and most preferably no more than about 10 seconds. Most preferably, the entire transport of the quantity of dialysate from probe 10 to measuring cell 14 is achieved in a single pulse of a pulsed flow pattern. This pulsed flow pattern is then repeated, each pulse adding fresh dialysate to the probe while bringing metabolite-rich dialysate from the probe to the measuring volume, and each diffusion period allowing diffusion of metabolite into the dialysate within the probe while the metabolite concentration in the sample reaching the measuring volume is determined.

It should be noted that this approach departs markedly from the continuous flow, or pseudo continuous flow, approach conventionally used in microdialysis probe systems. As detailed in the background section above, in a continuous flow system, the flow rate must be small enough to ensure sufficient dwell time of the dialysate within the probe. This inherently leads to slow transport of the sample to the measuring cell. Even in conventional pulsed flow systems, the pulse volume is typically small compared to the flow path volume, thereby giving rise to the same limitations as a continuous flow implementation. In contrast, according to this aspect of the present invention, a sample of dialysate is brought rapidly from the probe to the measuring cell for measurement of the metabolite content, thereby minimizing the delay between sampling and measurement, independent of the diffusion time needed for the dialysate within the probe to absorb metabolite from the surrounding tissue.

It should be noted that the required pulse volume is the flow volume required to displace part or all of the probe volume so as to deliver the required quantity of dialysate into the measuring volume. The “required quantity” is such that, when mixed in the desired proportions with reagent, the mixture of at least part of the fluid sample from the probe together with the reagent is sufficient to fill the measurement volume.

A further advantage of this aspect of the present invention is that the volume of dialysate used can be reduced. Specifically, in a pulsed system as described, a pulse need only be delivered when a measurement is required. Between pulses, the dialysate can remain within the probe for whatever diffusion period is desired, preferably approaching and/or maintaining equilibrium with the surrounding body fluids, ready for transport to the measuring volume by the next pulse. This contrasts clearly with continuous flow schemes where the response time of measurement and rate of dialysate usage are conflicting factors; dialysate flow rate can be reduced, but only at the cost of a slower response time in the measurements.

Regarding the pulsed flow of the invention, it will be clear that the required pulse volume may be subdivided into two or more pulses delivered in quick succession, or an extended pulse of significant duration, without departing from the scope of the invention, so long as the total transport time is short (preferably less than 25%, more preferably less than 10%, and most preferably no more than 5%) compared to the zero-flow diffusion period within the probe.

Thus, the flow pattern of this aspect of the present invention preferably includes: zero-flow diffusion periods during which the dialysate in the probe approaches equilibrium of the metabolite concentration with the surrounding body fluid; and transport flow periods during which sufficient flow is driven through the system to deliver a deliver the required quantity of dialysate into the measurement volume, mixed together with an appropriate quantity of a reagent if so needed. The transport flow periods are preferably less than 25%, more preferably less than 10%, and most preferably no more than 5% of the diffusion periods, and preferably take no more than half a minute, and more preferably within about 10 seconds.

As mentioned above, the various different aspects of the present invention described herein are believed to be of patentable significance each in its own right. However, it will be helpful here to point out certain aspects of particular synergy between these different aspects.

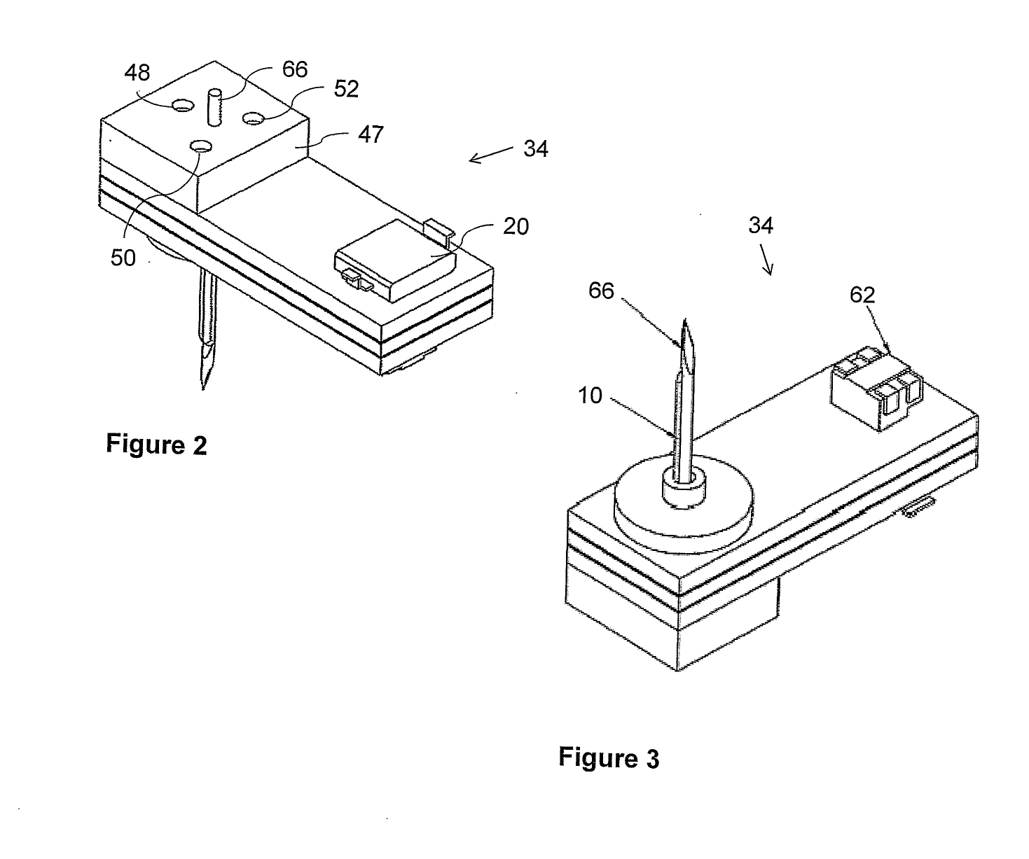

Firstly, the pulse volume required to bring the quantity of dialysate from the probe to the measuring cell according to the aforementioned aspect of the invention is a direct function of the volume of the flow path. In order to minimize the amount of dialysate used, it is clearly advantageous to minimize the volume of the flow path as much as possible. To this end, the layered device structure and associated probe structure described below with reference to FIGS. 2-7 is particularly advantageous.

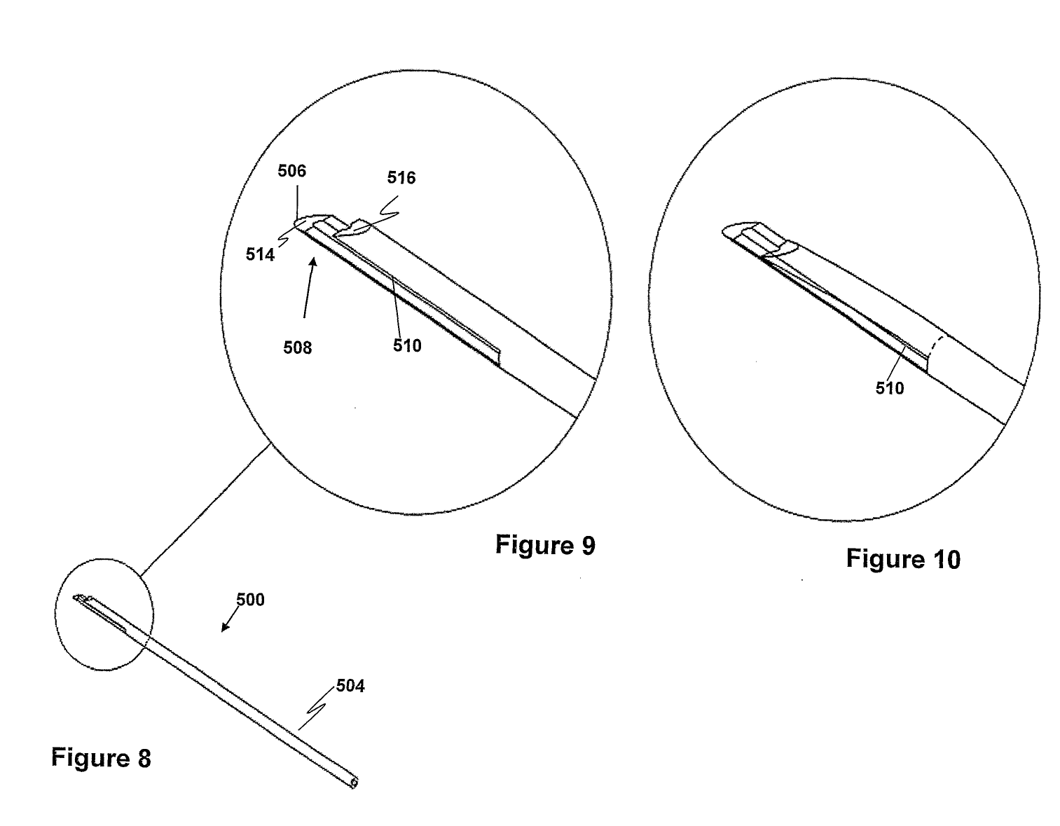

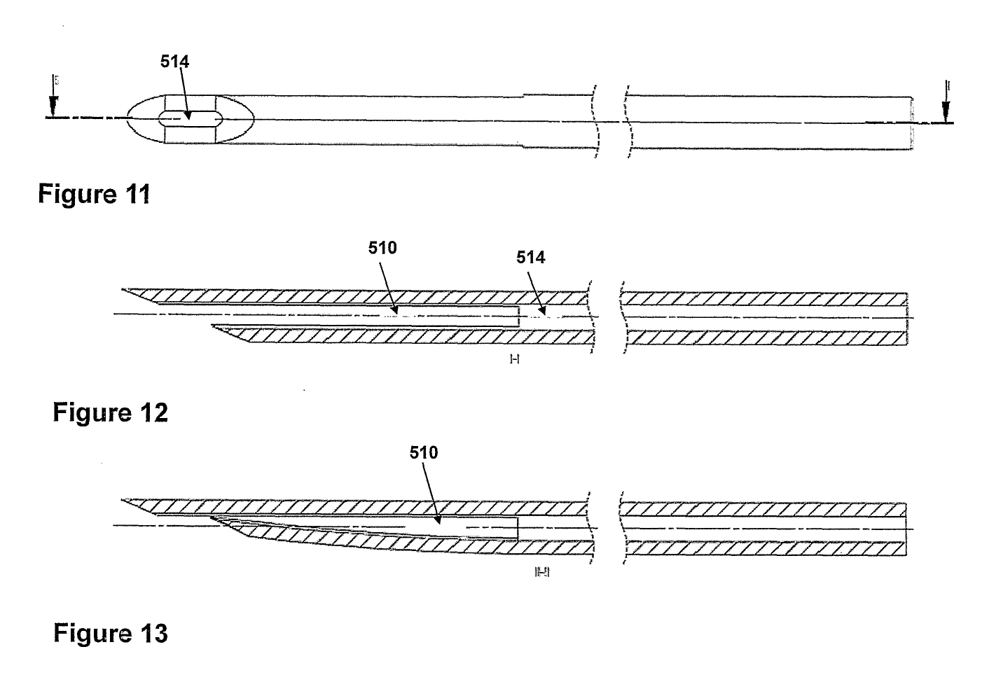

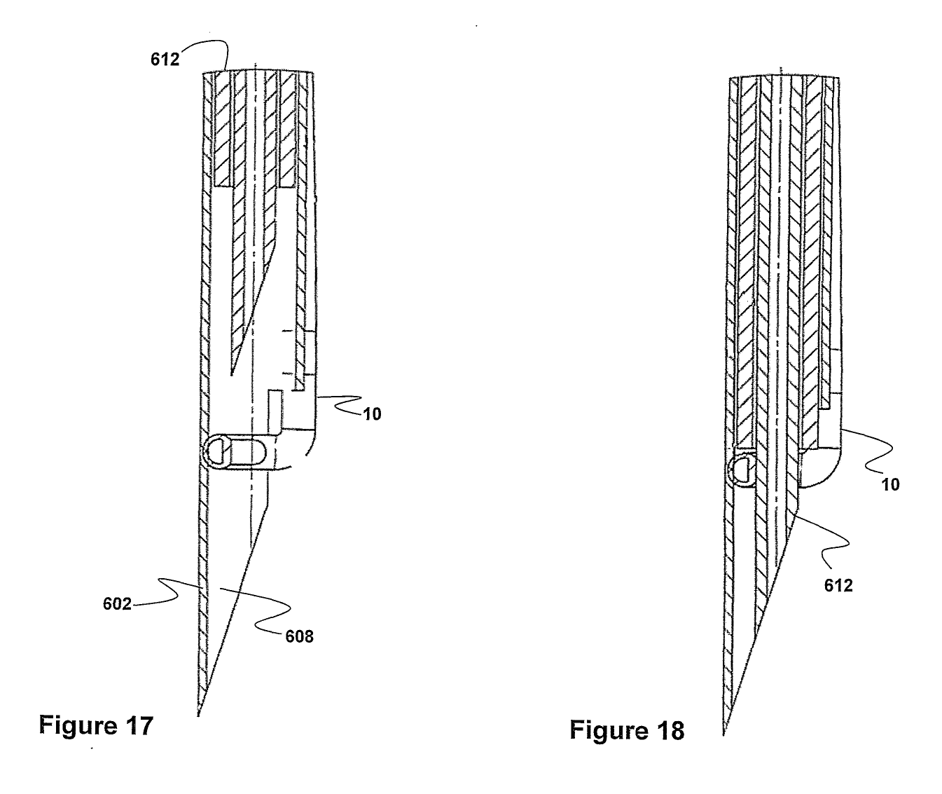

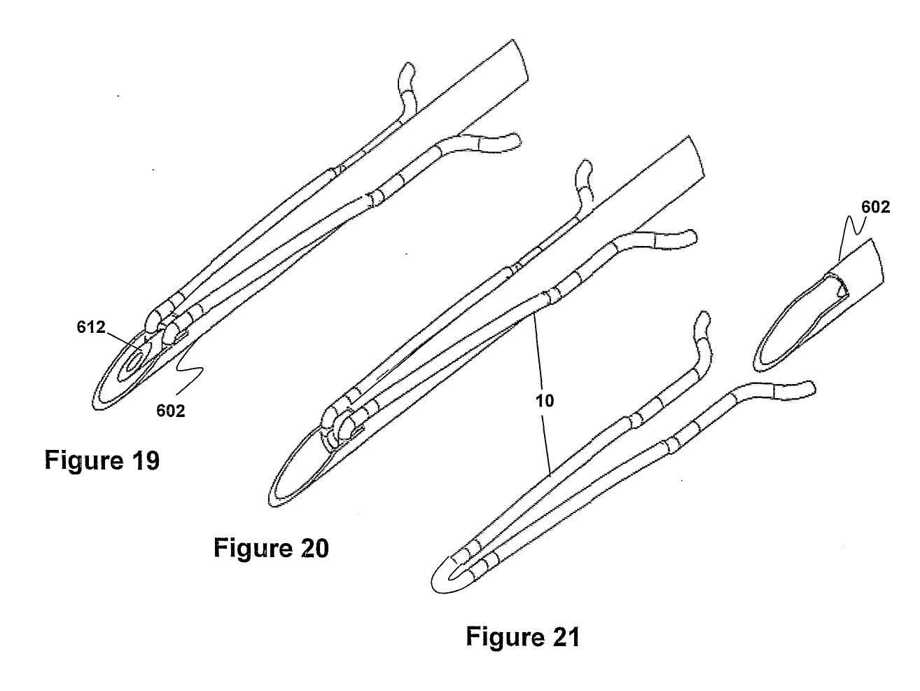

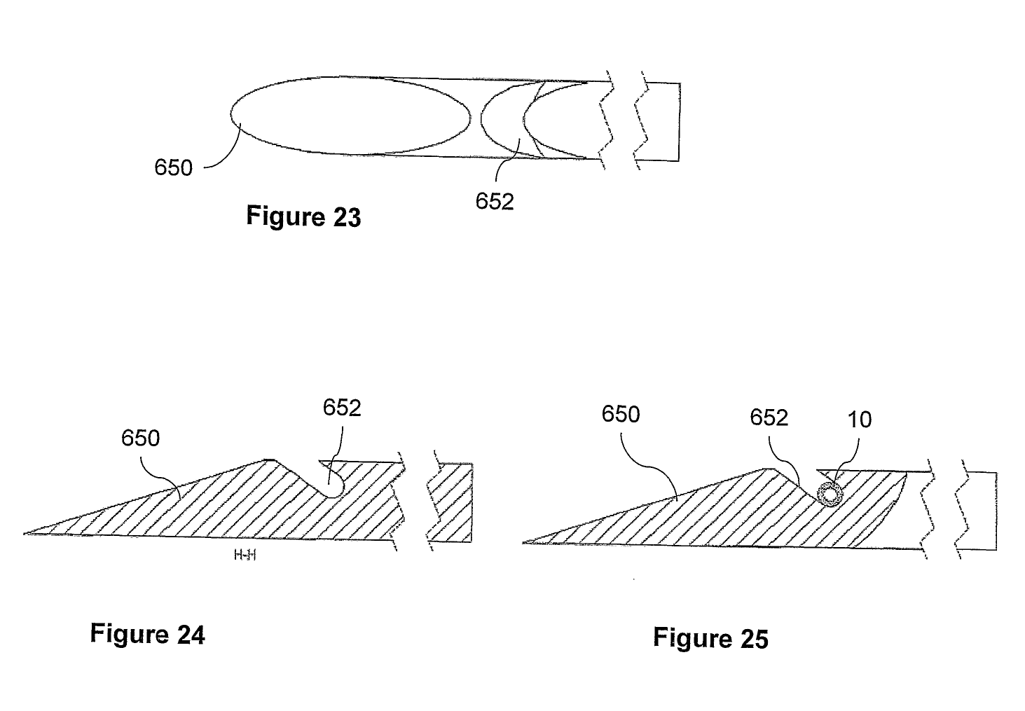

The probe structure of FIGS. 2-7 typically requires a corresponding method and device for deployment of the probe. Various preferred implementations of such a method and device are then discussed with reference to FIGS. 8-25.

Lastly, it will be noted that the rapid transport of the sample of dialysate from the probe to the measuring cell allows the use of measurement techniques based on the rate of change of an optical property of the fluid within the measuring cell. Such techniques are not typically feasible for continuous or rapidly pulsed systems. The use of rate-of-change measurements allows metabolite concentration to be derived before the reaction proceeds to completion, or even under conditions which do not provide enough reagent to reach completion, thereby further reducing the delay in measurement of the metabolite concentration.

The various additional aspects of the invention will now be described in turn.

### Layered Device Structure

According to a further aspect of the present invention, certain embodiments provide an implementation of a metabolite monitoring device in which connection of the microdialysis probe to the measurement system is achieved with very low volume and, in certain preferred implementations, such that the volume of the flow path between the probe catheter and the mixing channel of the measuring unit is no more than about 1 micro-liter. To this end, at least part of the flow path from the probe volume to the measuring volume is formed as a channel enclosed between at least two layers of a layered structure. Most preferably, no detachable connector is used between the probe and the measurement arrangement.

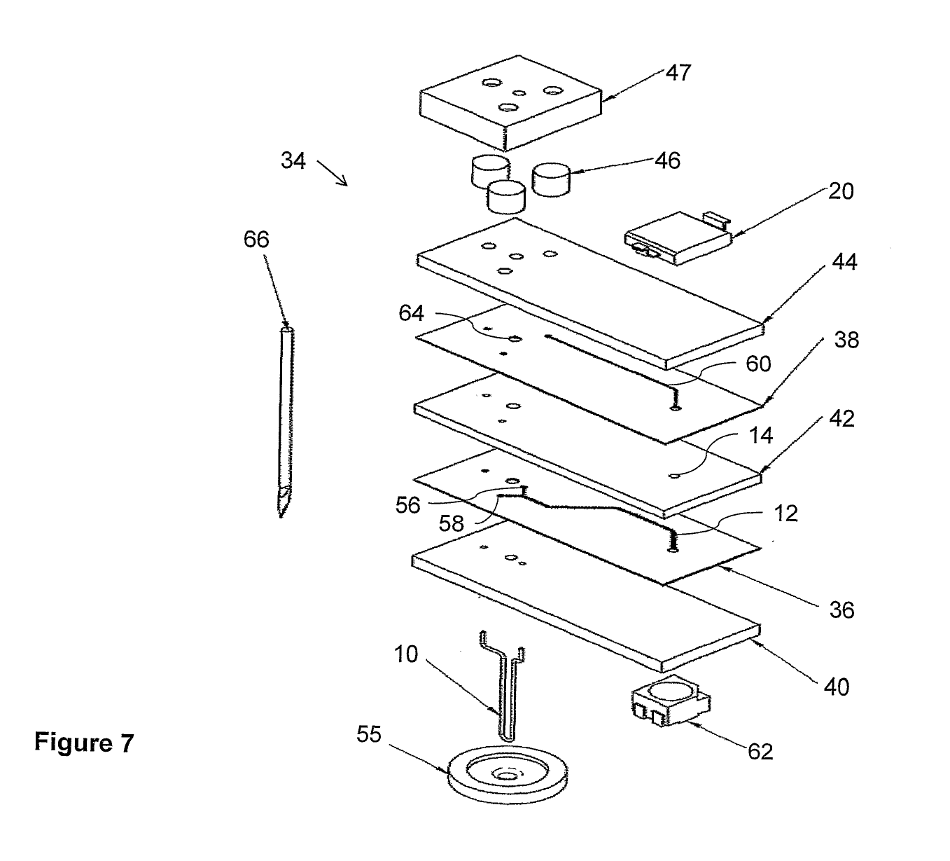

Turning now to FIGS. 2-7, there is shown an embodiment of a structure, generally designated 34, which illustrates particularly preferred features of a combined implementation of probe 10, flow path 12, measuring cell 14 and optical sensor 20. Referring particularly to the exploded view of FIG. 7, it can be seen that structure 34 is made up of a number of layers, in this case five, which define a pattern of flow channels. In the embodiment illustrated here, flow channels are defined by channels cut as through-slots in at least one channel layer 36, 38 and closed above and below by sealing layers 40, 42, 44. In an alternative embodiment, channels may be cut through only a part of the thickness of a channel layer and enclosed by addition of a single sealing layer, as will be clear to one ordinarily skilled in the art.

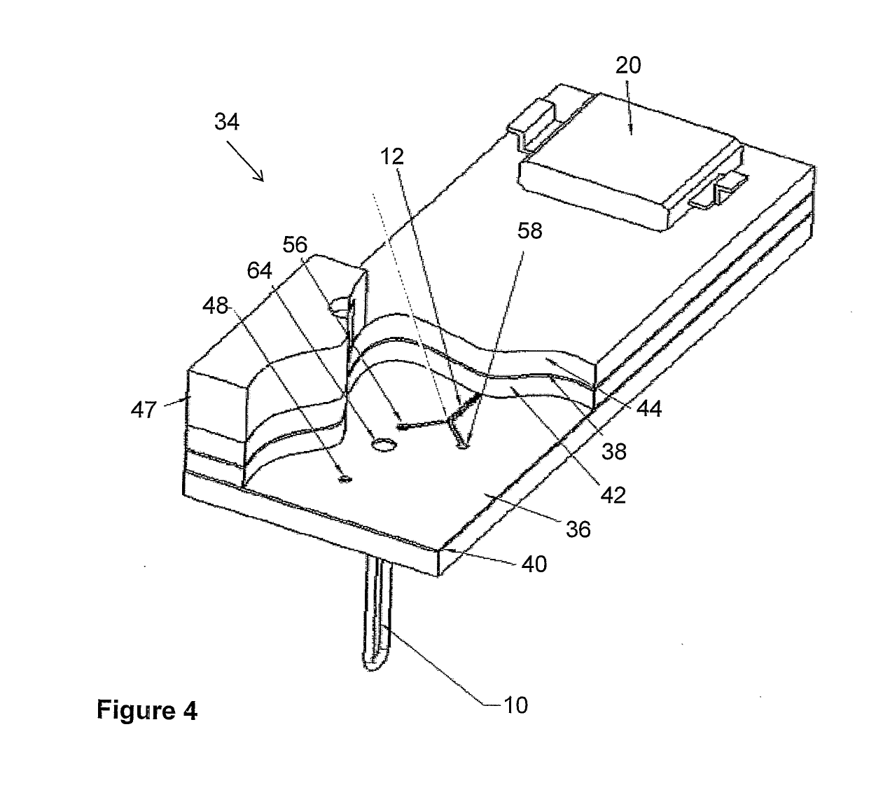

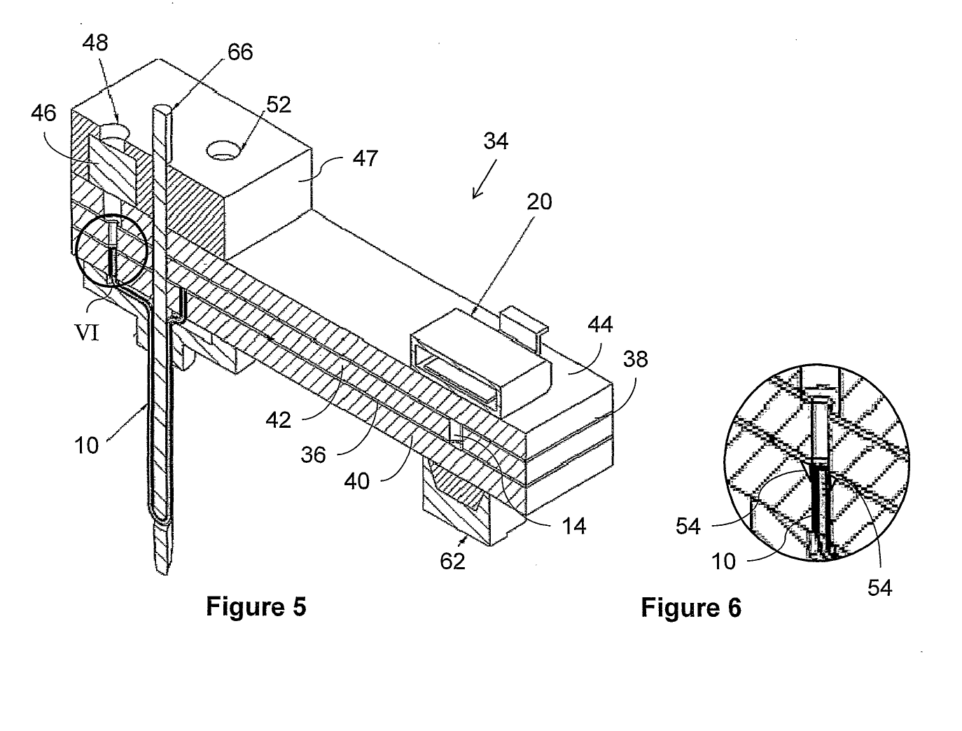

In the particular non-limiting example illustrated here, fluid connections to structure 34 are provided by three ports, each with a piercable septum seal 46 (visible in FIGS. 5 and 7) held in place by a port block 47. A first port 48 is the inlet for dialysate, a second port 50 is the inlet for reagent, and port 52 provides the outlet to the fluid dump.

As best seen in FIG. 5, from port 48, the dialysate passes through aligned holes in each layer to reach probe 10, which is implemented here as a fine hollow fiber (a length of flexible tube) formed primarily, and typically entirely, from a membrane permeable to the metabolite. Each end of the length of flexible tube is directly connected in a fluid-tight manner to layer 40. This is typically achieved by inserting an excess length of the probe tube extending upwards through holes in layer 40 and surrounding the base of the tube with a sealant-glue 54 (see FIG. 6). After setting of the glue, the excess length of tube is sliced off to leave the ends of probe 10 sealed to their respective apertures of layer 40. A lower sealing ring 55 is provided to offer some degree of support and protection to probe 10.

After dialysate has remained within the probe volume for a suitable diffusion period, the next pulse of dialysate to port 48 displaces the metabolite-rich dialysate through the connection of probe 10 to layer 40 at an aperture 56 (FIGS. 4 and 7) and along flow path 12. At the same time, the corresponding desired proportion of reagent is introduced through port 50 which connects through aligned apertures to a reagent supply channel 58 which delivers the reagent to mix with the dialysate within flow path 12. The combined mixture is displaced by the ongoing combined dialysate/reagent pulse until it reaches and fills measuring cell 14, enclosed between sealing layers 40 and 44. After measurement (to be described below), and when the diffusion period for the next sample in the probe has finished, the subsequent pulse of fluid flow carries the old sample from measuring cell 14 along an outflow channel 60 defined in layer 38 and sealed between layers 42 and 44 where it exits, either immediately or during a subsequent pulse, via outlet port 52.

As mentioned earlier, a particularly preferred technique for measuring the metabolite concentration is by optical sensing. In a preferred non-limiting implementation, absorption of light due to a color change between the metabolite and a reagent is detected. In the preferred implementation illustrated here, this optical sensing is achieved by implementing at least one of layers 40, 44 enclosing measuring volume 14 as a transparent layer, i.e., with a high proportion of transmission of optical radiation in at least one range of wavelengths relevant to measurement of the color change. In the case illustrated here, both layers 40 and 44 are implemented as transparent layers. An illumination source 62 (e.g., a LED) is deployed on the lower surface of layer 40 aligned with measuring cell 14, and optical sensor 20 is deployed on the upper surface of layer 44, also appropriately aligned.

It will be appreciated that structure 34 may be implemented using a wide range of different materials. The transparent layers may be various transparent polymer layers, or glass layers. Channel layers 36 and 38 may advantageously be formed from material which is readily machined or molded to provide the required fine channels, and may be various sorts of polymer or metal. Clearly, the materials should be inert to the dialysate, the reagent and the metabolite, and should be appropriate for use in a medical device. However, other than the probe itself and other base surfaces likely to contact the skin, the materials may not need to be fully “biocompatible.”

As should already be clear, probe 10 is designed to be deployed as a free-standing probe within the body tissue of the subject during operation of the metabolite monitoring device. Various inserters suitable for deploying the hollow fiber probe within the body will be discussed below. In order to facilitate their use, layered structure 34 preferably provides an insertion bore 64 aligned with probe 10 for insertion of an inserter, shown schematically as an inserter needle 66 in FIGS. 2, 3, 5 and 7, to support the probe during insertion into the body of a subject.

### Probe Inserters

As discussed above, a preferred embodiment of a metabolite monitoring device described herein employs a stand-alone, flexible probe 10 deployed in the tissue of a subject. Probe 10 is inserted through the skin into the tissue of the subject by way of a stiff inserter configured to support probe 10 during insertion and to leave probe 10 inserted to a desired deployment depth after withdrawal of the insertion mechanism. To facilitate correct deployment of probe 10, certain preferred embodiments of the invention employ an inserter which combines an elongated shaft having a penetrating tip and a recess for receiving a part of said probe, with an actuator element, deployed at least partially coextensively with said elongated shaft. The elongated shaft and the actuator element are preferably configured such that, in a first relative position of the elongated shaft and the actuator element, the inserter retains the probe for insertion through a biological barrier into tissue, and, when the actuator element is displaced relative to the elongated shaft, the probe is released, thereby allowing withdrawal of the inserter from the tissue while the probe remains inserted within the tissue.

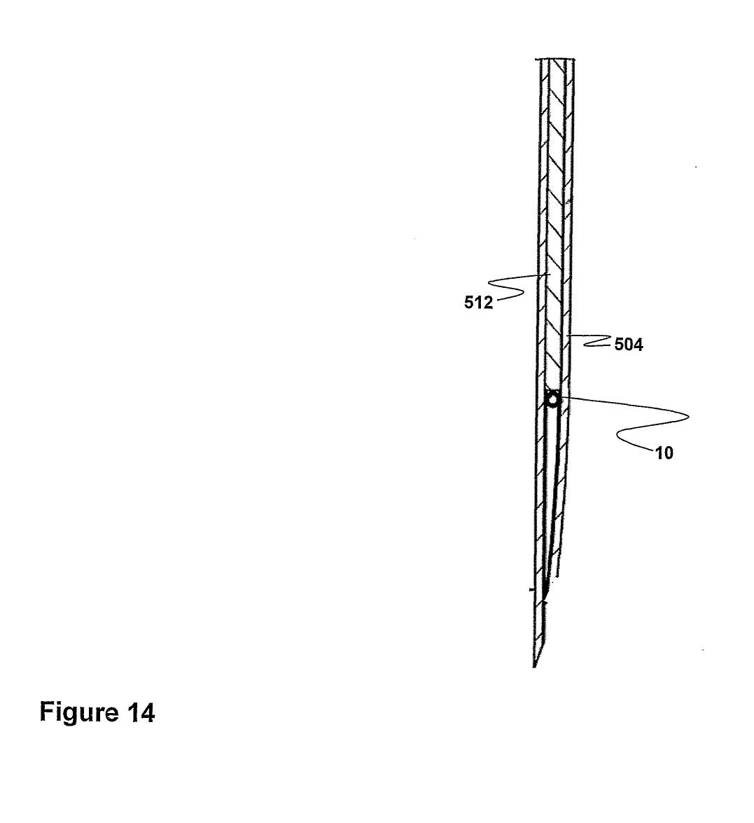

FIGS. 8 through 14 depict a first non-limiting, exemplary embodiment of an insertion mechanism 500 including an elongated shaft 504 implemented as a tube, a penetrating tip 506 for piercing the skin and tissue, a beveled end 508, a slot 510 for receiving probe 10 (most clearly shown in FIG. 14) and a push rod 512, serving as the actuator element for actuating the release of probe 10 from insertion tube 504. Penetrating tip 506, in a non-limiting exemplary embodiment, is implemented as a cutting edge formed by a single bevel, but it should be appreciated that a cutting edge formed by a double bevel or a puncturing point formed by a taper are also included within the scope of any embodiment of the current invention. Slot 510 is disposed across beveled end 508 so as to divide beveled end 508 into two beveled surfaces, 514 and 516. In a non-limiting embodiment, the portion of shaft 504 on the side of slot 510 supporting beveled surface 514 is resiliently biased to bear on the other part of shaft 504 that supports beveled surface 516. This biasing avoids catching tissue in slot 510 during penetration into the body tissue of a patient, and also helps to retain probe 10 within slot 510 prior to deployment. Clearly, a similar effect can be achieved by biasing the second portion against the first, or by biasing both portions together. Slot 510 is disposed in a manner that preserves the structural integrity of piecing tip 510 and has a depth of three to four millimeters in a non-limiting embodiment. Push rod 512 is disposed in the inserter tube lumen 514 in a manner providing freedom of axial movement.

Prior to deployment, probe 10 is inserted into slot 510, where it is retained by the closing together of two sides of slot 510 and/or by maintaining slight tension in the probe fiber. Insertion mechanism 500 carrying probe 10 is then inserted into the subject's body, either manually or by any suitable automated insertion mechanism.

Upon achieving the desired deployment depth, push rod 512 is advanced axially relative to shaft 504 (either by advancing push rod 512 or withdrawing shaft 504, or by a combination of these motions) so as to open apart the two sides of slot 510 and urge probe 10 out of the slot. After probe 10 is thus disengaged from slot 510, shaft 504 can be further retracted and withdrawn from the tissue together with, or followed by, push rod 512, without drawing probe 10 after them.

It should be noted that the aforementioned sequence of motions may be performed manually by suitable manipulation of the rear portions of shaft 504 and push rod 512, or can be mechanized by any suitable mechanism, as will be clear to one ordinarily skilled in the art.

In a non-limiting exemplary embodiment, insertion tube 504 may be formed from a 26 or 27-gauge, stainless-steel hypodermic needle, suitably processed to form slot 510 and impart the resilient bias described above, while push rod 512 is implemented as a suitably sized pin, typically of similar material, inserted within the lumen of the hollow needle.

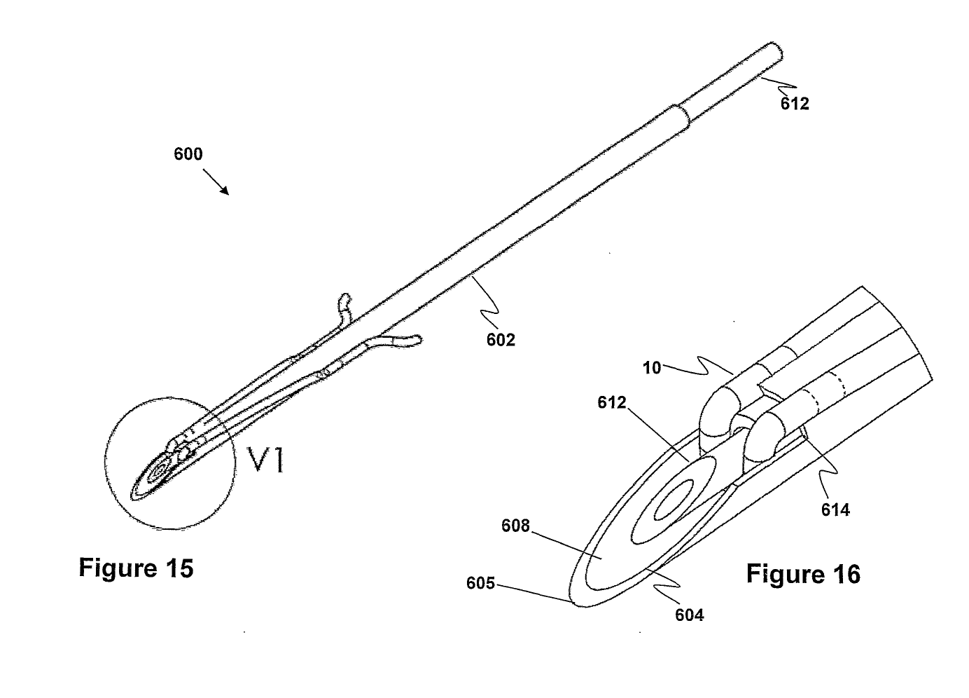

Referring now to FIGS. 15-21, these depict a second, non-limiting exemplary embodiment of a probe inserter 600 that includes an insertion tube 602 having an insertion tube penetrating tip 605, and a holding rod 612 for retaining probe 10, having a holding rod penetrating tip 606. Holding rod 612 is disposed in lumen 608 in a manner providing axial freedom of motion and operative to secure and release probe 10 inside lumen 608.

Prior to deployment, the distal loop of probe 10 is inserted into lumen 608 by way of an opening in insertion tube wall 614, as most clearly visible in FIG. 16. Holding rod 612 is displaced within lumen 608 so as to secure probe 10 inside insertion tube 602 as shown in FIGS. 15-17. Holding rod 612 is preferably provided with a low friction spacer sleeve as shown which helps maintain holding rod 612 centered within insertion tube 602. This configuration is maintained while insertion tube insertion tube 602 penetrates the subject's body to the desired tissue depth. During insertion into the body, the remaining lengths of probe 10 are disposed in a substantially parallel orientation to insertion tube 602; each length being disposed on opposite sides of insertion tube 602 to minimize drag and the possibility of probe damage. After insertion tube 602 has achieved the desired tissue depth, relative axial displacement of insertion tube 602 and holding rod 612 releases the retention of probe 10, as shown in FIG. 20. Insertion tube 602 and holding rod 612 can then be sequentially withdrawn without dragging probe 10 after them, thus leaving probe 10 deployed in the desired position and depth within the patient's body (FIG. 21).

It should be noted that holding rod 612 may optionally be implemented as a solid shaft. Most preferably, the end of holding rod 612 is implemented with a beveled tip as shown to complement the beveled tip portion of insertion tube 602, thereby helping to avoid punching out of a block of tissue by the insertion tube during penetration. It will be appreciated however that holding rod 612 need not exhibit a sharp point, and that various alternative forms of the holding rod may be employed to achieve a similar function.

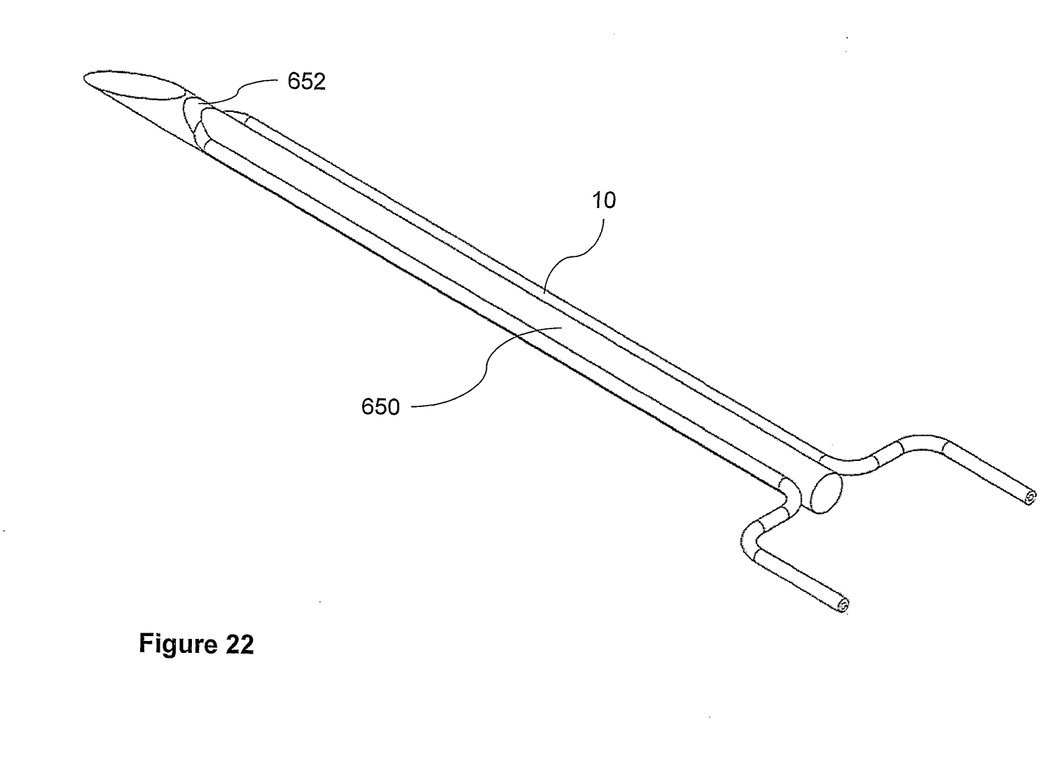

Finally regarding the inserter, although the aforementioned examples of inserters with retention and release mechanisms are believed to be particularly advantageous, it should be noted that a simple single-element inserter used to insert probe 10 into the body of a subject also falls within the scope of various aspects of the present invention. FIGS. 22-25 depict a non-limiting example of a single-element inserter 650 suitable for use according to the teachings of the present invention. With suitable shaping of the recess 652 which supports probe 10 during insertion, axial rotation of inserter 650 can be additionally helpful for releasing probe 10 from the recess so that inserter 650 can be withdrawn leaving probe 10 deployed within the tissue.

### Rate-of-Change Measurements

The present invention discloses an additional feature directed towards converting optical parameter measurements of the fluid in the measuring cell into metabolite content values on the basis of the rate of reaction of the metabolite and the reagent

It should be noted at this point that traditional systems, by necessity, perform the above conversion on the basis of a final concentration as opposed to the disclosed rate of reaction. This necessity is a consequence of traditional flow arrangements in which the system fluids are in a state of continuous flow or effective continuous flow resulting from repeated flow pulses with short time delays between each pulse. A given fluid volume subjected to a measuring illumination at a first given moment is not the identical fluid volume subjugated to a second measuring illumination a moment later because each volume is flowing through the measuring cell. Optical measurements based on rates of change require a single volume of sample be subjugated to the measuring illumination at two different stages of the reaction. The disclosed pulsed flow system leaves a single sample within measuring cell 14 for the diffusion period between pulses, therefore allowing enough time to perform multiple measurements of the changing optical properties of the dialysate-reagent mixture as the reaction progresses, and hence allowing metabolite concentration to be determined based on a rate of reaction.

The use of rate of change of optical properties for determining the metabolite concentration may in some implementations provide significant advantages. For example, the use of rate of reaction may enable more rapid determination of the measurement of metabolite concentration, since it is not necessary to wait for the reaction to go to completion. Furthermore, the rate of reaction can be used to determine metabolite concentration even in a case where the reagent is not present in sufficient excess to allow all of the metabolite to react. This allows certain particularly preferred implementations of the present invention to use a reduced quantity of reagent in proportion to the dialysate compared with the theoretically prescribed quantity for full-reaction measurements of the concentration. Such reduced quantities are of great practical importance, further reducing the size of the fluid reservoirs which must be carried in a body-mounted metabolite monitoring device.

Thus, in practical terms, a particular non-limiting exemplary implementation of this aspect of the present invention preferably allows optical measurements to be performed less than one minute after mixing the dialysate sample with the reagent, thereby eliminating the customary ten minutes required for complete metabolite reaction associate with traditional systems. Furthermore, since the measurements are performed before the metabolite completely reacts with the reagent; the quantity of reagent fed into the system is reduced from 100 times the sample volume to about 25 times the sample volume, thereby greatly facilitating system miniaturization.

A fixed quantity of about 25 times the sample volume of reagent is mixed with the sample volume upon entry into measuring cell 14. A glucose color reagent that produces a color having around a 505 nm wavelength is employed in an exemplary, non-limiting embodiment. Such a reagent is commercially available from RAICHEM (San Diego, Calif.), as catalogue number 80038.

Referring now to FIG. 5, the fluid in measuring cell 14 is illuminated by electromagnetic radiation in the VIS-NIR range generated by illumination source 62. Optical sensor 20 measures the radiation that passes through the sample. The total volume of the dialysate and the reagent mixture and their proportions are constant so that the only significant non-fixed factor affecting the kinetics of the color producing reaction is the glucose content in the dialysate. In an exemplary, non-limiting embodiment, processor 30 applies an algorithm to the transmittance data to derive glucose, or any other metabolite, value as will be further discussed

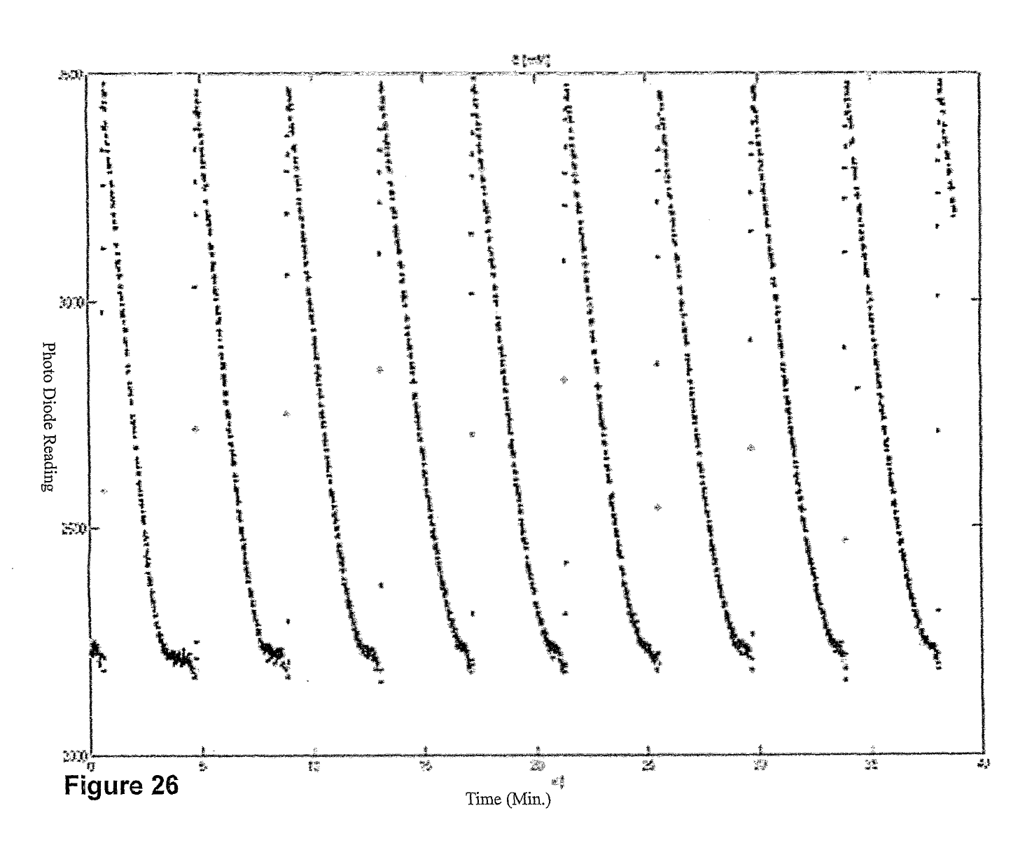

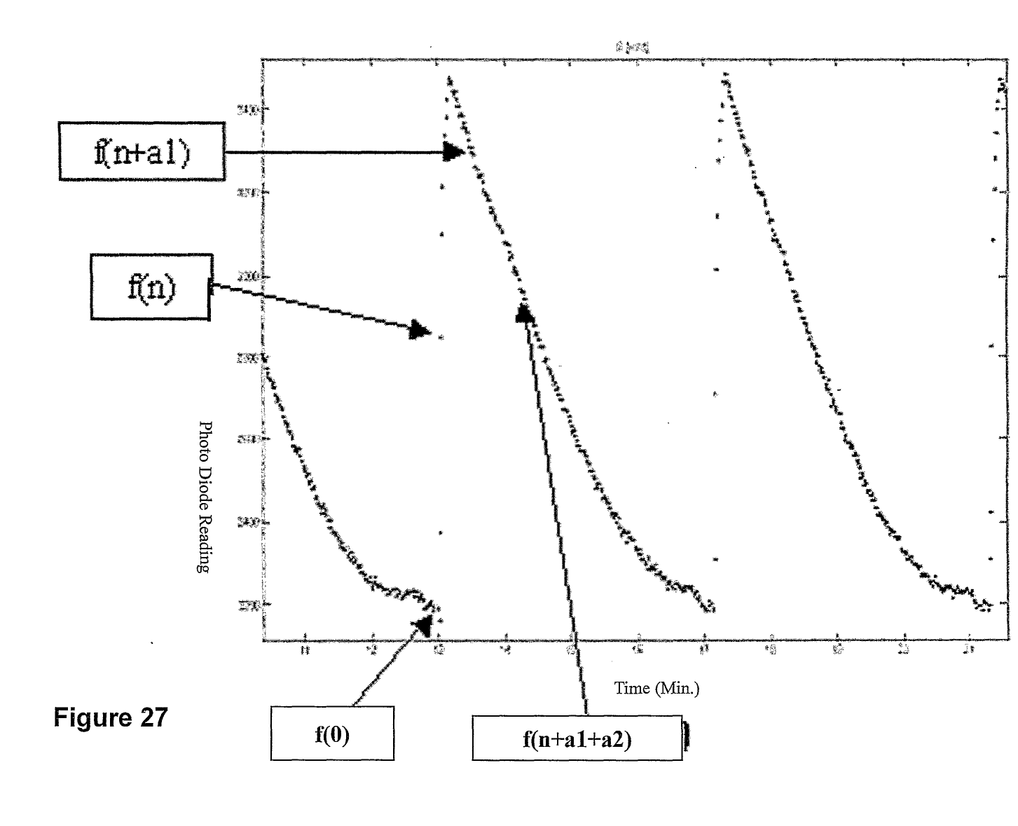

FIGS. 26 and 27 are transmittance charts depicting the amount of radiant energy passing through the dialysate/reagent mixture as a function of time for each discrete volume of sample conveyed into measuring cell 14. As shown, in the non-limiting, exemplary embodiment, a new sample is conveyed into measuring cell 14 every four minutes and optical sensor 20 is configured to measure transmittance every second. As the metabolite reacts with the reagent, the color of the mixture darkens and the transmittance decreases. The rate of change of transmittance is indicative of a corresponding concentration of the metabolite content; greater metabolite contents generate correspondingly more rapid rates of change.

In general terms, the metabolite concentration determination is performed by identifying arrival of a new fluid sample to be measured, deriving representative transmittance values, calculating a rate of change of transmittance, and transforming the rate of change of transmittance into a glucose content value.

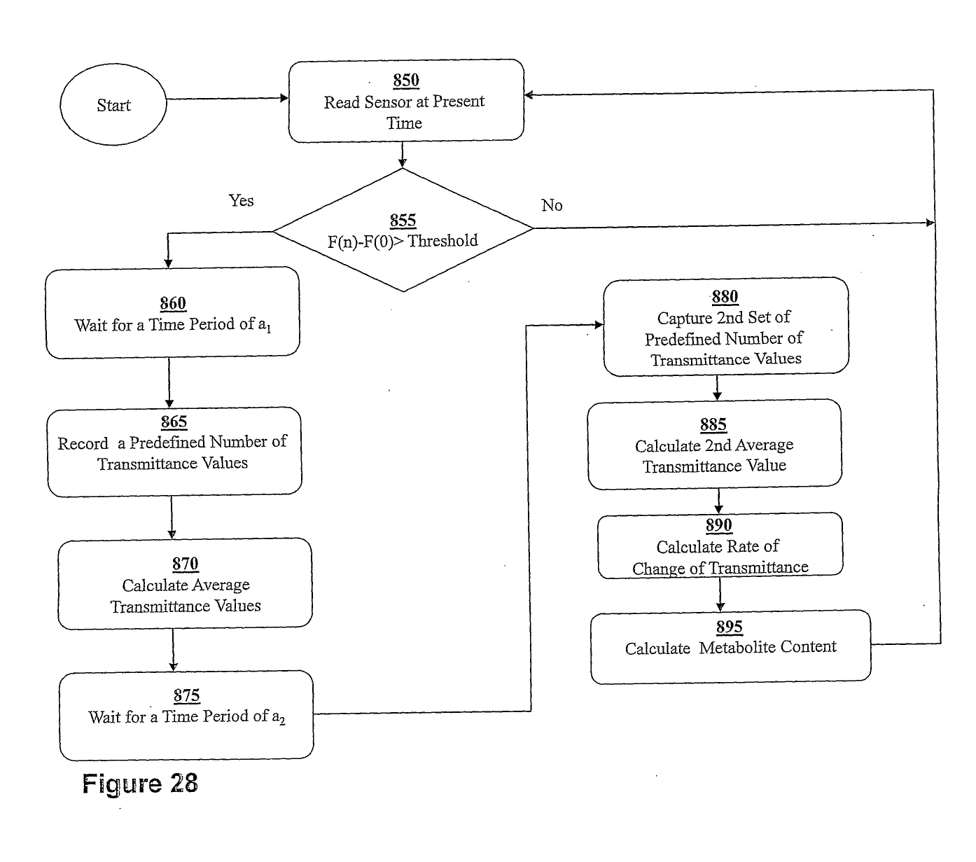

Referring now to FIGS. 27 and 28, the transmittance value at point f(0) represents the transmittance value at the completion of a series of measurements performed for a prior sample. The sudden increase in transmittance represented by point(s) f(n) indicate that a new sample of dialysate/reagent mixture has reached the measuring cell 14. Thus, in the flow diagram of FIG. 28, the algorithm measures the transmittance (step 850) and checks if the difference in transmittance between f(0) and f(n) exceeds a predefined threshold (step 855), thereby initiating a new measurement cycle. When a new measurement cycle is initiated, the algorithm begins timing 860 a delay period a₁ to ensure that the replacement of the old sample with the new sample has been completed. At the end of delay period a₁, the algorithm records at 865 a predetermined number of transmittance measurements and calculates and records at 870 their average value, and then begins a second timing period a₂ (step 875). At the end of second delay period a₂ the algorithm records a second set of a predefined number of transmittance values at step 880, and calculates and records the average value their average value at 885. Based on these two average values, the algorithm calculates a rate of change of transmittance 890. The algorithm then employs the measured rate of changing to derive the body fluid metabolite concentration (step 895) based on transmittance and metabolite relationships known to those skilled in the art, typically in combination with data from an initial calibration procedure performed prior to use, again as well known in the art.

It will be noted that the aforementioned technique for determining the rate of change of transmittance is merely one particular example, and that other techniques, such as best-fit of a polynomial function to the measurements, may be used. Furthermore, the algorithm may readily be adapted to different situations, including a reflective optical arrangement.

It will be appreciated that the above descriptions are intended only to serve as examples, and that many other embodiments are possible within the scope of the present invention as defined in the appended claims.

1. A method for monitoring the concentration of a metabolite in vivo within a body fluid, the method comprising the steps of:
(a) providing a monitoring device comprising:
(i) a microdialysis probe formed at least in part from a membrane permeable to the metabolite, the probe defining a probe volume containing dialysate,
(ii) a measuring cell having a measuring volume, and
(iii) a flow path connecting from the probe volume to the measuring volume;
(b) bringing the probe into contact with the body fluid;
(c) maintaining substantially zero flow conditions during a diffusion period to allow diffusion of the metabolite into the dialysate within the microdialysis probe; and
(d) generating a dialysate flow to carry a quantity of dialysate from the probe volume along the flow path to the measuring volume,
wherein said generating a dialysate flow is performed such that said quantity of dialysate passes from the probe volume to the measuring volume in a given transport time, said transport time being no more than 25 percent of said diffusion period.

2. A device for monitoring the concentration of a metabolite in vivo within a body fluid, the device comprising:
(a) a microdialysis probe for bringing into contact with the body fluid, said probe being formed at least in part from a membrane permeable to the metabolite, said probe defining a probe volume containing dialysate;
(b) a measuring cell having a measuring volume;
(c) a flow path connecting from the probe volume to the measuring volume; and
(d) a fluid flow controller deployed to control flow of dialysate through said probe volume and said flow path to said measuring volume, said fluid flow controller being configured to generate a pulsed flow pattern including:
(i) a diffusion period of substantially zero flow conditions to allow diffusion of the metabolite into the dialysate within said probe; and
(ii) a dialysate flow to carry a quantity of dialysate from the probe volume along said flow path to the measuring volume,
wherein said flow controller generates said dialysate flow such that said quantity of dialysate passes from the probe volume to the measuring volume in a given transport time, said transport time being no more than 25 percent of said diffusion period.

3. The method of claim 1, wherein said transport time is no more than 10 percent of said diffusion period.

4. The method of claim 1, wherein said substantially zero flow conditions and said dialysate flow are generated repeatedly as part of a pulsed flow pattern, and wherein a single fluid pulse of said pulsed flow pattern transports said quantity of dialysate from the probe volume to the measuring volume.

5. The method of claim 1, wherein at least part of the flow path from the probe volume to the measuring volume is formed as a channel enclosed between at least two layers of a layered structure.

6. The method of claim 5, wherein said layered structure further comprises a reagent inlet in flow connection with said flow path for introducing a reagent for mixing with the dialysate from said probe prior to the dialysate reaching said measuring volume.

7. The method of claim 5, wherein said measuring volume is enclosed between layers of said layered structure.

8. The method of claim 7, wherein at least one of said layers enclosing said measuring volume is a transparent layer, and wherein the device further comprises an optical sensor for sensing an optical parameter of fluid within said measuring volume via said at least one transparent layer.

9. The method of claim 5, wherein said probe comprises a length of flexible tube formed primarily from said membrane permeable to the metabolite, each end of said length of flexible tube being in fluid connection with an aperture formed in at least one layer of said layered structure.

10. The method of claim 9, wherein said layered structure further comprises an insertion bore aligned with said probe for insertion of an inserter to support said probe during insertion into the body of a subject.

11. The method of claim 9, wherein the device further comprises an inserter comprising:
(a) an elongated shaft having a penetrating tip and a recess for receiving a part of said probe; and
(b) an actuator element, deployed at least partially coextensively with said elongated shaft,
wherein said elongated shaft and said actuator element are configured such that, in a first relative position of said elongated shaft and said actuator element, said inserter retains said probe for insertion through a biological barrier into tissue, and, when said actuator element is displaced relative to said elongated shaft, said probe is released, thereby allowing withdrawal of said inserter from the tissue while said probe remains inserted within the tissue.

12. The method of claim 1, wherein the device further comprises:
(a) an optical sensor deployed for sensing an optical parameter of fluid within said measuring volume; and
(b) a concentration calculator operationally connected to the optical sensor, the concentration calculator comprising at least one processor, the concentration calculator being configured to derive a rate of change of the optical parameter of the fluid within said measuring volume under substantially zero flow conditions, and to calculate a concentration of the metabolite within the body fluid based at least in part on said rate of change.

13. A device for monitoring the concentration of a metabolite in vivo within a body fluid, the device comprising:
(a) a microdialysis probe for bringing into contact with the body fluid, said probe being formed at least in part from a membrane permeable to the metabolite, said probe defining a probe volume containing dialysate;
(b) a measuring cell having a measuring volume; and
(c) a flow path connecting from the probe volume to the measuring volume,
wherein at least part of the flow path from the probe volume to the measuring volume is formed as a channel enclosed between at least two layers of a layered structure.

14. The device of claim 13, wherein said layered structure further comprises a reagent inlet in flow connection with said flow path for introducing a reagent for mixing with the dialysate from said probe prior to the dialysate reaching said measuring volume.

15. The device of claim 13, wherein said measuring volume is enclosed between layers of said layered structure.

16. The device of claim 15, wherein at least one of said layers enclosing said measuring volume is a transparent layer, and wherein the device further comprises an optical sensor for sensing an optical parameter of fluid within said measuring volume via said at least one transparent layer.

17. The device of claim 13, wherein said probe comprises a length of flexible tube formed primarily from said membrane permeable to the metabolite, each end of said length of flexible tube being in fluid connection with an aperture formed in at least one layer of said layered structure.

18. The device of claim 17, wherein said layered structure further comprises an insertion bore aligned with said probe for insertion of an inserter to support said probe during insertion into the body of a subject.

19. The device of claim 17, wherein the device further comprises an inserter comprising:
(a) an elongated shaft having a penetrating tip and a recess for receiving a part of said probe; and
(b) an actuator element, deployed at least partially coextensively with said elongated shaft,
wherein said elongated shaft and said actuator element are configured such that, in a first relative position of said elongated shaft and said actuator element, said inserter retains said probe for insertion through a biological barrier into tissue, and, when said actuator element is displaced relative to said elongated shaft, said probe is released, thereby allowing withdrawal of said inserter from the tissue while said probe remains inserted within the tissue.

20-22. (canceled)

23. The device of claim 2, wherein said transport time is no more than 10 percent of said diffusion period.

24. The device of claim 2, wherein said substantially zero flow conditions and said dialysate flow are generated repeatedly as part of a pulsed flow pattern, and wherein a single fluid pulse of said pulsed flow pattern transports said quantity of dialysate from the probe volume to the measuring volume.

25. The device of claim 2, wherein at least part of the flow path from the probe volume to the measuring volume is formed as a channel enclosed between at least two layers of a layered structure.

26. The device of claim 25, wherein said layered structure further comprises a reagent inlet in flow connection with said flow path for introducing a reagent for mixing with the dialysate from said probe prior to the dialysate reaching said measuring volume.

27. The device of claim 25, wherein said measuring volume is enclosed between layers of said layered structure.

28. The device of claim 27, wherein at least one of said layers enclosing said measuring volume is a transparent layer, and wherein the device further comprises an optical sensor for sensing an optical parameter of fluid within said measuring volume via said at least one transparent layer.

29. The device of claim 25, wherein said probe comprises a length of flexible tube formed primarily from said membrane permeable to the metabolite, each end of said length of flexible tube being in fluid connection with an aperture formed in at least one layer of said layered structure.

30. The device of claim 29, wherein said layered structure further comprises an insertion bore aligned with said probe for insertion of an inserter to support said probe during insertion into the body of a subject.

31. The device of claim 29, wherein the device further comprises an inserter comprising:
(a) an elongated shaft having a penetrating tip and a recess for receiving a part of said probe; and
(b) an actuator element, deployed at least partially coextensively with said elongated shaft,
wherein said elongated shaft and said actuator element are configured such that, in a first relative position of said elongated shaft and said actuator element, said inserter retains said probe for insertion through a biological barrier into tissue, and, when said actuator element is displaced relative to said elongated shaft, said probe is released, thereby allowing withdrawal of said inserter from the tissue while said probe remains inserted within the tissue.

32. The device of claim 2, wherein the device further comprises:
(a) an optical sensor deployed for sensing an optical parameter of fluid within said measuring volume; and
(b) a concentration calculator operationally connected to the optical sensor, the concentration calculator comprising at least one processor, the concentration calculator being configured to derive a rate of change of the optical parameter of the fluid within said measuring volume under substantially zero flow conditions, and to calculate a concentration of the metabolite within the body fluid based at least in part on said rate of change.
